(19) United States
(12) Patent Application Publication
(10) Pub. No.: US 2011/0137058 A1
(43) Pub. Date: Jun. 9, 2011

(54) PREPARATION OF PARICALCITOL

(75) Inventors: CHZE-SIONG NG, TAOYUAN CITY (TW); CHING-PENG WEI, TAOYUAN CITY (TW)

(73) Assignee: FORMOSA LABORATORIES, INC., TAOYUAN CITY (TW)

(21) Appl. No.: 13/027,657

(22) Filed: Feb. 15, 2011

Related U.S. Application Data
(62) Division of application No. 12/112,856, filed on Apr. 30, 2008.

(51) Int. Cl. C07C 401/00

(52) U.S. Cl. 552/653

(57) ABSTRACT

This invention relates to a method for purifying Paricalcitol by reverse phase chromatography. This invention also relates to a purified Paricalcitol prepared by said method. This invention further relates to a method for purifying Paricalcitol by crystallization.

### CROSS-REFERENCE TO RELATED APPLICATIONS

This application is a Divisional of the pending U.S. patent application Ser. No. 12/112,856 filed on Apr. 30, 2008, all of which is hereby incorporated by reference in its entirety.

Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of the entire prior art of record and any search that the Office deems appropriate.

### FIELD OF THE INVENTION

This invention relates to a method for purifying Paricalcitol by reverse phase chromatography. This invention also relates to a purified Paricalcitol prepared by said method. This invention further relates to a method for purifying Paricalcitol by crystallization.

### DESCRIPTION OF PRIOR ART

The 19-nor vitamin analogue, Paricalcitol(I), is characterized by the following formula:

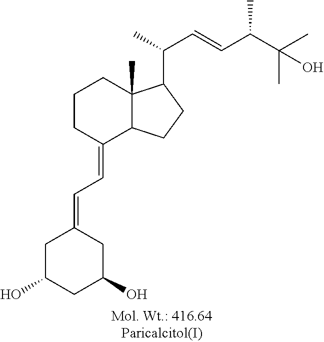

In the synthesis of vitamin D analogues, a few approaches to obtain a desired active compound have been outlined previously. One of the methods is the Wittig coupling attachment of a 1α,3β-Bis(tert-Butyldimethylsiloxy)-(20s)-(diphenylphosphonium)-19-nor secoergosterol-5(Z),7(E)-diene to a key intermediate PCT-S3 to obtain the desired Paricalcitol, as shown in U.S. patent application Ser. No. 11/953,527.

During the preparation of Paricalcitol, various unwanted by-products may be formed, and which kind of by-product may be formed depends on the method for its preparation. One of the most comment by-products is its C-24 isomer.

The synthesis of Paricalcitol requires many synthetic steps; unfortunately those steps produce undesired by-products. Therefore, the final product may be contaminated not only with a by-product derived from the last synthetic step of the process but also with compounds formed in previous steps. However, in the United States, the Food and Drugs Administration guidelines recommend that the amounts of some impurities should be limited to less than 0.1 percent. Thus, the purification of Paricalcitol is a long-time issue.

Residual solvents in pharmaceuticals are defined as organic volatile chemicals that are used or produced in the manufacture of drug substances or excipients, or in the preparation of drug products. Since the solvents can not be completely removed by the practical manufacturing techniques, the content of solvents in these products should be evaluated and justified. In the ICH guideline (Q3C, impurities: Guideline for residual solvents) recommends use of less toxic solvents and there are certain guidelines indicating the amount of solvents which can be remained in the products for each solvent.

Since there are no therapeutic benefit form residual solvents, all residual solvents should be removed to the extent which meets product specifications, good manufacturing practices, or other quality-based requirements. The level of residual solvent in drug product should be lower than the safety standard. Solvents associated with less severe toxicity (Class 2, solvents such as acetonitrile (no more than 410 ppm) and methyl chloride (no more than 600 ppm) should be limited in order to protect patients from potential adverse effects. Ideally, less toxic solvents such as class 3 solvents, including 2-propanol, n-heptane and ethyl acetate, which have PDEs of 50 mg or more per day should be used where practicable. Therefore, it is important to reduce the residual solvents impurities in final the products.

Some methods for the preparation of 19-nor vitamin D analogue are described in U.S. Pat. No. 5,281,731 and U.S. Pat. No. 5,086,191. However, in these patents, normal phase preparative HPLC is the only chromatography used, and it is used for preparation of 1α, 25-dihydroxy-vitamin D3 (U.S. Pat. No. 5,281,731, Zorbax sil. 9.4×25 cm column, mobile phase: 20% 2-propanol in hexane) and 1α,22-dihydroxy-19-nor-vitamin D (U.S. Pat. No. 5,086,191, Zorbax sil. 9.4×25 cm column, mobile phase: 10% ethyl acetate in hexane), but not Paricalcitol [(7E,22E)-19-Nor-9,10-Secoergosta-5,7,22-triene-1α,3β,25-triol]. In addition, normal phase preparative HPLC had fallen out of favor in the 1970's because of a lack of reproducibility of retention times as water or organic solvents changed the hydration state of the silica or alumina chromatographic media.

Other methods for Paricalcitol preparation such as crystallization methods are described in U.S. Pub. No. 2,007,149,489 and U.S. Pub. No. 2,007,093,458. In these applications, the yield of crystallization is about 50˜80%. However, the solvent used for the preparation of Paricalcitol by the disclosed crystallization method is tert-butanol, therefore the crystalline paricalcitol is a tert-butanol solvate which contains more than 1% undesirable tert-butanonl. Therefore, even though the yield of Paricalcitol is 60% and the purity is 99.63%, the residual solvent impurity is still a problem.

In U.S. Pub. No. US 2,007,093,458, the initial ratio of Paricalcitol and crystallization solvent, is higher than 1:150 g/ml which render the purity of the Paricalcitol hardly meets the USP requirement for Paricalcitol related substance. The guideline requires the purity of Paricalcitol related substance to be at least 99.5%, the greatest impurity to be no more than 0.1% and the total impurity no more than 0.5%.

For a long time, the manufactures of Paricalcitol constantly faces the needs of high yield of medicinal substances with high chromatographic purity, low production cost and a favorable ecological balance. Unfortunately, the preparation of Paricalcitol in present can not fulfill the needs. For example, U.S. Pub. No. 2,007,149,489 discloses a method for the purification of Paricalcitol by crystallization. In that method, the cooling temperature for crystallization is below −10° C. due to the nature of the solvent and crystallization process. Since the low temperature and the rate of cooling are difficult to control, the mount of residual solvent often result in more than 1% impurity. Moreover, because the proportion of impurity in crude Paricalcitol is quite high, purification by said method would result in high cost.

### BRIEF DESCRIPTION OF THE DRAWINGS

FIG. 1 illustrates the synthesis of Paricalcitol as described in U.S. patent application Ser. No. 11/953,527.

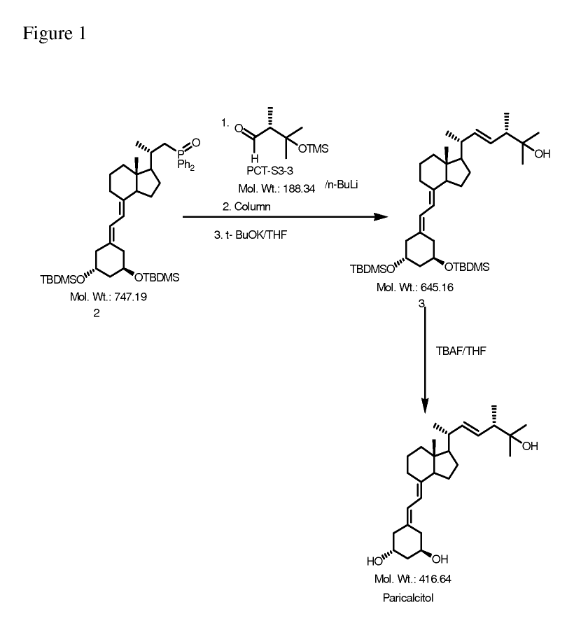

FIG. 2 shows the XRD spectrum of crystal Paricalcitol.

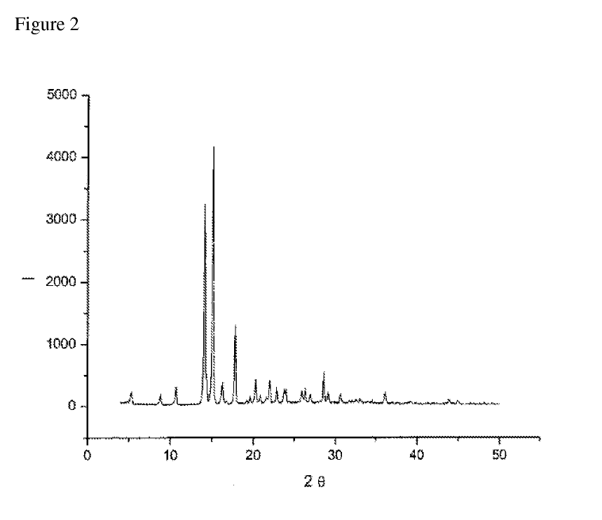

FIG. 3 shows chromatograph for purity of Paricacitol Crude.

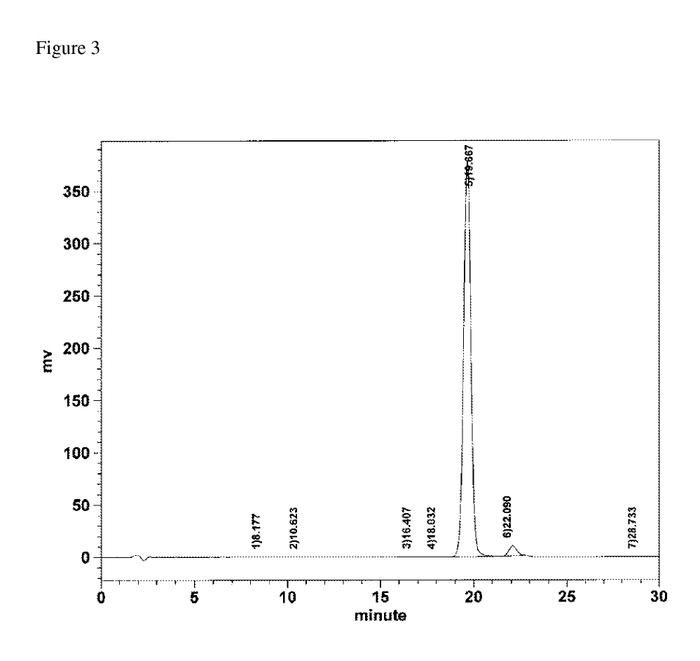

FIG. 4 shows the Prep-HPLC chromatograph for the purity of Paricacitol Crude.

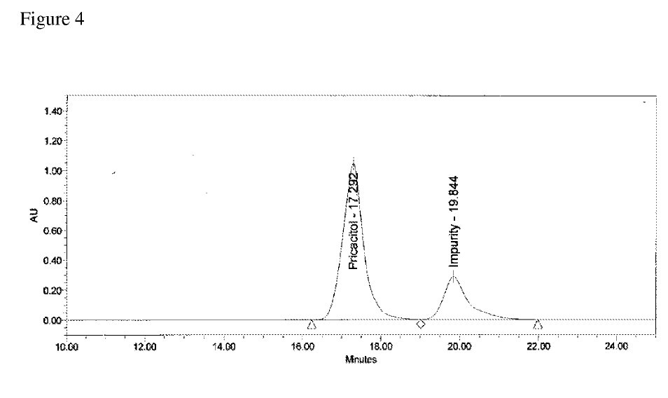

FIG. 5 shows the HPLC chromatograph of purified Paricacitol.

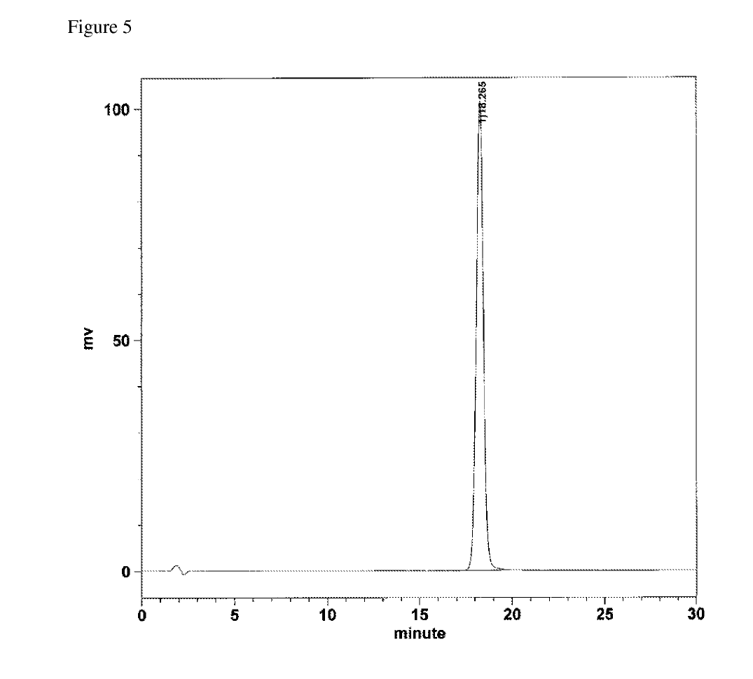

### SUMMARY OF THE INVENTION

The present invention provides a method for purifying Paricalcitol which comprises:
(a) dissolving Paricalcitol-crude in a solvent;
(b) adding the dissolved Paricalcitol-crude into a chromatography column;
(c) conditioning a chromatography column with a mobile phase selected from the group consisting of organic solvent, buffer and water;
(d) collecting fractions comprising Paricalcitol; and
(e) removing the organic solvent by concentration and filtration to give Paricalcitol.

The present invention also provides a Paricalcitol, made by said method, which has at least 99% purity which meets the Food and Drugs Administration guidelines in the United States.

The present invention further provides a method for purifying Paricalcitol which comprises:

(a) dissolving Paricalcitol in a solvent for crystallization to form a solution;
(b) cooling the solution to form a precipitate;
(c) filtering precipitate; and
(d) drying the precipitate with vacuum to give pure Paricalcitol.

### DETAILED DESCRIPTION OF THE INVENTION

The high yield, low cost and high purity of Paricalcitol with diminished impurity and unwanted by-product are highly demanded for the manufactures.

The present invention provides a method for purifying Paricalcitol, which comprise:
(a) dissolving Paricalcitol-crude in a solvent;
(b) adding the dissolved Paricalcitol-crude into a chromatography column;
(c) conditioning a chromatography column with a mobile phase selected from the group consisting of organic solvent, buffer and water;
(d) collecting fractions comprising Paricalcitol; and
(e) removing the organic solvent by concentration and filtration to give Paricalcitol.

In a preferred embodiment, the present invention further comprises:
(a) dissolving Paricalcitol in step (e) mentioned above in a solvent for recrystallization to form a solution;
(b) cooling the solution to form a precipitate;
(c) filtering the precipitate; and
(d) drying the precipitate with vacuum to give pure Paricalcitol.

In a preferred embodiment, the mobile phase consists of 55% acetonitrile in water or buffer.

Preferably, the solvent for dissolving Paricalcitol-crude is C₁-C₄ alcohol, C₁-C₆ ether, cyclic ether or dimethyl sulfoxide (DMSO).

More preferably, the solvent for dissolving Paricalcitol-crude is methanol, 2-propanol or DMSO.

The solvent for recrystallization of the present invention is preferably selected from the group consisting of alcohol, water, ester and alkane; provided that the solvent excludes alcohol or ester alone.

More preferably, the alcohol is methanol or 2-propanol; the ester is ethyl acetate; and the alkane is heptane.

The present invention produces pure Paricalcitol at a rate ranging from 20 mg/per hour ˜200 mg/per hour.

In a preferred embodiment, the solution is cooled at a temperature ranging from 0˜25° C.

More preferably, the temperature ranges from 5˜20° C.

The method of the present invention further comprises a stationary phase as a reverse phase made of natural or synthetic crosslinked polymer.

In a preferred embodiment, the natural polymer is silica gel with alkyl chains of different lengths.

Preferably, the synthetic crosslinked polymer consists of styrene and divinylbenzene.

Preferably, the stationary phase has particle size ranges from 1 μm to 900 μm.

In addition, the stationary phase of the present invention can be regenerated with 20˜100% of a lower alcohol or a lower alcohol in water or acetonitrile or acetonitrile in water solution after the chromatography is completed.

The present invention further provides a purified Paricalcitol with at least 99% purity, and said Paricalcitol is prepared by said method.

Most preferably, the purity of said Paricalcitol is at least 99.5% purity.

The present further provides a method for purifying Paricalcitol which comprises:

(a) dissolving Paricalcitol in a solvent for crystallization to form a solution;
(b) cooling the solution to form a precipitate;
(c) filtering precipitate; and
(d) drying the precipitate with vacuum to give pure Paricalcitol.

The solvent of said method is preferably selected from the group consisting of alcohol, water, ester and alkane; provided that the solvent excludes alcohol or ester alone. Preferably, the alcohol is C₁-C₄ alcohol; the ester is C₂-C₆ ester; and the alkane is C₁-C₈ alkane. More preferably, the alcohol is methanol or 2-propanol. Most preferably, the ester is ethyl acetate, and the alkane is heptane.

### EXAMPLE

The examples below are non-limiting and are merely representative of various aspects and features of the present invention.

### Example 1

### Purification of Paricalcitol

Experimental data for displacement chromatography are as follows:

The Paricalcitol crude purity was around 97% and the total impurities were 3.0%.

The stationary phase was an octadecyl silica gel column 50×200 mm (reverse phase, XBridge™ Prep C18, 5 nm OBD™ Waters Inc.) with a particle size of 5 μm.

The mobile phase with a flow rate of 110 mL/min consisted of 55% acetonitrile in water.

The entering crude Paricalcitol (13.7 g) had a concentration of 50 mg/mL of methanol.

The capacity of the process was 100 mg of sample per hour.

The total yield of the obtained product was 88%. The product was separated into two fractions, if necessary, the other fraction being repeatedly purified.

The suitable fraction was concentrated to remove the organic solvent, after concentration to obtain Pure Paricalcitol (purity of 99.9%).

The Pure Paricalcitol was dried at 28° C. under vacuum (P˜2 mmHg) for 48 hours, to give 13.7 g crystalline Paricalcitol (the residual solvent impurities: acetonitrile: 1219 ppm).

TABLE 1
Chromatograph data for purity of Paricalcitol crude.

| | | Area | | Height % | |
|---|---|---|---|---|---|
| No. | RT (min) | (UV* sec) | Area | (mv) | Height % |
| 1 | 8.177 | 1160 | 0.0110 | 0.0579 | 0.0149 |
| 2 | 10.623 | 1126 | 0.0107 | 0.0656 | 0.0169 |
| 3 | 14.407 | 8093 | 0.0767 | 0.3570 | 0.0917 |
| 4 | 18.032 | 7165 | 0.0679 | 0.3016 | 0.0775 |
| 5 | 19.667 | 10255270 | 97.1416 | 378.5816 | 97.2666 |
| 6 | 22.090 | 280180 | 2.6540 | 9.7360 | 2.5014 |
| 7 | 28.733 | 4040 | 0.0383 | 0.1209 | 0.0311 |
| | Total | 10557034 | | 389.221 | |

TABLE 2
Peak result of prep-HPLC Chromatograph data for the purity
of Paricalcitol Crude

| | | | | % |
|---|---|---|---|---|
| | Name | RT | Area | Area |
| 1 | Pricalcitol | 17.292 | 38950625 | 75.57 |
| 2 | Impurity | 19.844 | 12590343 | 24.43 |

TABLE 3
HPLC Chromatograph data of purified Paricalcitol

| | | Area | | Height % | |
|---|---|---|---|---|---|
| No. | RT (min) | (UV* sec) | Area | (mv) | Height % |
| 1 | 18.265 | 2719470 | 100.000 | 101.4826 | 100.000 |
| | Total | 2719470 | | 101.483 | |

TABLE 4

| | acetonitrile | |
|---|---|---|
| Item | (NMT 410 ppm)* | Remarks |
| Residual solvents | 1219 ppm | Out of the ICH |
| | | guideline |

*ICH guideline recommends acetonitrile is a class II solvent, the safely limit is NMT 410 ppm.

### Example 2

The Paricalcitol crude purity was around 97% and the impurities were 3.0%.

Sample preparation: 1 g Crude Paricalcitol in 25 mL methanol or DMSO.

The stationary phase was an octadecyl silica gel column 19×100 mm (reverse phase, Sunfire™ Prep C18, 5 μm OBD™ Waters Inc.) with a particle size of 5 μm.

The mobile phase with a flow rate of 110 mL/min consisted of 55% acetonitrile in water. The entering crude Paricalcitol (1 g) had a concentration of 50 mg/mL of Methanol. The capacity of the process was 100 mg of sample per hour.

The total yield of the obtained product was 75%. The product was separated into two fractions, if necessary, the other fraction being repeatedly purified.

The suitable fraction was concentrated to remove the organic solvent, after concentration, pure Paricalcitol (purity of 99.5%) was obtained.

### Example 3

The Paricalcitol crude purity was around 97% and the impurities were 3.0%.

Sample preparation: 1 g Crude Paricalcitol in 25 mL methanol.

The stationary phase was an octadecyl silica gel column 19×100 mm (reverse phase, Atlantis™ Prep C18, 5 μm OBD™ Waters Inc.) with a particle size of 5 μm.

The mobile phase with a flow rate of 110 mL/min consisted of 55% acetonitrile in water. The entering crude Paricalcitol (1 g) had a concentration of 50 mg/mL of Methanol. The capacity of the process was 50 mg of sample per hour.

The total yield of the obtained product was 80%. The product was separated into 2 fractions with two fraction, if necessary, the other fraction being repeatedly purified.

The suitable fraction was concentration to remove the organic solvent, after concentration, pure Paricalcitol (purity of 99.70%) was obtained.

### Example 4

The Paricalcitol crude purity was around 97% and the impurities were 3.0%.

Sample preparation: 1 g Crude Paricalcitol in 25 mL methanol.

The stationary phase was an octadecyl silica gel column 19×100 mm (reverse phase, Atlantis™ Prep C18, 5 μm OBD™ Waters Inc.) with a particle size of 5 nm.

The mobile phase with a flow rate of 110 mL/min consisted of 55% acetonitrile in buffer solution which is prepared by a solution containing 1.0 g/L of tris(hydroxymethyl)aminomethane adjusted to pH 7.25±0.25 with phosphoric acid.

The entering crude Paricalcitol (1 g) had a concentration of 50 mg/mL of methanol.

The capacity of the process was 50 mg of sample per hour.

The total yield of the obtained product was 80%. The product was separated into two fraction, if necessary, the other fraction being repeatedly purified.

The suitable fraction was concentration to remove the organic solvent, after concentration, pure Paricalcitol (purity of 99.70%) was obtained.

### Example 5

### Crystallization of Paricalcitol from Methanol/Ethyl Acetate/N-Heptane

130 mg Paricalcitol (obtained from preparative of Prep-HPLC, before drying) were dissolved in 5.0 mL 50% methanol in ethyl acetate mixtures, at 30° C., with stirring, during 30 minutes. The clear solution was filtered through glass wool into another flask, and 13 mL n-heptane was added. The solution was then concentrated by evaporation to a volume of 5 mL of solution mixtures (control by weight). The solution was cooled to 5° C., and that temperature was maintained 5 minutes. The crystals were filtered and washed with 13 mL of cold n-heptane, and then dried at high vacuum in an oven at 28° C. for 48 hours to obtain a yield of 125 mg (purity of 99.90%, any other individual impurity NMT 0.10%). The residual solvent impurities testing results can meet the ICH guideline.

The residual solvent impurities were analysis by GC, the results is shown in Table 5.

TABLE 5

| | | | | ethyl | |
|---|---|---|---|---|---|
| | | n-heptane | methanol | acetate | |
| | acetonitrile | (NMT | (NMT | (NMT | |
| | (NMT 410 | 5000 | 3000 | 5000 | |
| Item | ppm)* | ppm)* | ppm)* | ppm)* | Remarks |
| Residual | ND | 4292 ppm | 980 ppm | 664.7 ppm | Meets the |
| solvents | | | | | ICH |
| | | | | | guideline |

*ICH Guideline

### Example 6

### Crystallization of Paricalcitol from 2-Propanol/Purified Water

10.3 g Paricalcitol (obtained from preparative of Prep-HPLC, before drying) were dissolved in 608 mL 2-propanol, at 35±5° C., with stiffing, during 10 minutes. Then, the solution was filtered through glass wool to another flask to obtained Paricalcitol-2-Propanol solution.

The Paricalcitol-2-Propanol solution was slowly added to stirring water (1160 mL) at 35±5° C. The solution was cooled to 15-20° C. (room temperature), and maintained for 3 hours. Then, the obtained crystalline material was filtered, and dried at 28° C. under vacuum (P-2 mmHg) for 24 hours, to give 9.41 g crystal Paricalcitol (purity of 99.95%, any other individual impurity NMT 0.10%).

The residual solvent was analysis by GC, the results is shown in Table 6.

TABLE 6

| | acetonitrile | 2-propanol | |
|---|---|---|---|
| Item | (NMT 410 ppm)* | (NMT5000 ppm)* | Remarks |
| Residual | ND | 3070 ppm | Meets the ICH |
| solvents | | | guideline |

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

What is claimed is:

1. A method for purifying Paricalcitol which comprises:
(a) dissolving Paricalcitol-crude in a solvent;
(b) adding the dissolved Paricalcitol-crude into a chromatography column;
(c) conditioning a chromatography column with a mobile phase;
(d) collecting fractions comprising Paricalcitol;
(e) removing the organic solvent by concentration and filtration to give Paricalcitol;
(f) dissolving Paricalcitol in step (e) in a solvent for recrystallization to form a solution;
(g) cooling the solution at a temperature ranging from 0-25° C. to form a precipitate;
(h) filtering the precipitate; and
(i) drying the precipitate with vacuum to give pure Paricalcitol.

2. The method of claim 1, wherein the mobile phase consists of 55% acetonitrile in water.

3. The method of claim 1, wherein the solvent for dissolving Paricalcitol-crude is C₁-C₄ alcohol.

4. The method of claim 1, wherein the solvent for dissolving Paricalcitol-crude is methanol.

5. The method of claim 1, wherein the solvent for recrystallization is 2-propanol and water.

6. The method of claim 1, which produces pure Paricalcitol at a rate ranging from 20 mg/per hour˜200 mg/per hour.

7. The method of claim 1, wherein the temperature ranges from 5˜20° C.

8. The method of claim 1 which further comprises a stationary phase as a reverse phase made of natural or synthetic crosslinked polymer.

9. The method of claim 8, wherein the natural polymer is silica gel with alkyl chains of different lengths.

10. The method of claim 8, wherein the synthetic crosslinked polymer consists of styrene and divinylbenzene.

11. The method of claim 8, wherein the stationary phase has particle size ranges from 1 μm to 900 μm.

12. The method of claim 8, wherein the stationary phase is regenerated with 20˜100% of a lower alcohol or a lower alcohol in water or acetonitrile or acetonitrile in water solution after the chromatography is completed.
